(19) United States
(12) Patent Application Publication
(10) Pub. No.: US 2018/0318415 A1
(43) Pub. Date: Nov. 8, 2018

(54) NOVEL ADJUVANT FORMULATIONS

(71) Applicant: GLAXOSMITHKLINE BIOLOGICALS, SA, Rixensart (BE)

(72) Inventors: David BURKHART, Hamilton, MT (US); Jay T. Evans, Hamilton, MT (US); Hardeep OBEROI, Hamilton, MT (US); Yvonne M. YORGENSEN, Hamilton, MT (US)

(73) Assignee: GLAXOSMITHKLINE BIOLOGICALS, SA, Rixensart (BE)

(21) Appl. No.: 15/781,351

(22) PCT Filed: Dec. 6, 2016
(86) PCT No.: PCT/EP2016/079954

§ 371 Date: Jun. 4, 2018

Related U.S. Application Data
(60) Provisional application No. 62/264,506, filed on Dec. 8, 2015.

(51) Int. Cl. A61K 39/39; A61K 39/145

(52) U.S. Cl. CPC A61K 39/39; A61K 2039/55572; A61K 39/145

(57) ABSTRACT

The invention relates to a composition comprising solid lipid nanoparticles (SLNs), wherein the SLNs comprise an aminoalkyl glucosaminide phosphate (AGP).

### STATEMENT REGARDING FEDERALLY SPONSORED RESEARCH

Aspects of this invention were made with the United States government support pursuant to contract #HHSNHHSN272200900008C from the National Institutes of Health; the United States government may have certain rights in the invention.

### FIELD OF THE INVENTION

The present invention relates to novel adjuvant formulations and methods for the preparation thereof, as well as to uses thereof. In particular, the invention relates to novel formulations of aminoalkyl glucosaminide phosphates (AGPs).

### BACKGROUND OF THE INVENTION

Toll-like receptors (TLRs) are linked to the innate immune response and recognise distinct structural components that are unique to pathogens. Agonists of TLRs are being used in medicine, for example as adjuvants in vaccines in order to potentiate immune responses. The first microbial product discovered to be a Toll-like receptor agonist was LPS, a bacterial membrane component specific to Gram-negative bacteria, which activates TLR4. Although LPS is a potent immunomodulatory agent, its medicinal use is limited due to its extreme toxicity.

AGPs are a different class of compounds which interact with TLR4, as agonists or antagonists. AGPs include both acyclic and cyclic compounds and have been described e.g. in U.S. Pat. No. 6,113,918, U.S. Pat. No. 6,303,347, WO 98/50399, WO 01134617, WO 01190129, WO 02112258 and WO 2004062599. These compounds have been demonstrated to retain significant adjuvant characteristics when formulated with antigens in vaccine compositions. AGPs also demonstrate mucosal adjuvant activity and are effective in the absence of antigen, making them attractive compounds for prophylactic and/or therapeutic use.

While AGPs have improved toxicity profiles when compared with LPS or derivatives thereof, their use in pharmaceutical settings will be benefited by further improvement of the toxicity profile, including less pyrogenic formulations.

### SUMMARY OF THE INVENTION

In a first aspect, the invention relates to a composition comprising solid lipid nanoparticles (SLNs), wherein the SLNs comprise an aminoalkyl glucosaminide phosphate (AGP).

In a second aspect, the invention relates to an immunogenic composition comprising an SLN composition as described herein and an antigen.

In a further aspect, the invention relates to a composition as described herein, or an immunogenic composition as described herein, for use in medicine, such as for use in the immunisation of a human subject.

In an even further aspect, the invention relates to a method for preparing a composition comprising SLNs as described herein, said method comprising:
a. Dissolving and mixing AGP and a lipid in an organic solvent
b. Removing the organic solvent from the mixture
c. Heating the resulting mixture and mixing with an aqueous solution containing a surfactant to generate an oil-in-water mixture, and
d. Performing a size reduction step, for example using microfluidization or sonication, and
e. Allowing the composition to cool
   wherein method optionally includes a filtration step.

### FIGURES

FIG. 1: Cationic lipids/surfactants used in preparation of cationic SLNs

FIG. 2: Post-secondary serum IgG titers (top), post-tertiary serum IgG titers (middle) and post-tertiary tracheal/vaginal wash IgA titers (bottom; vaginal wash—black bars, tracheal wash—grey bars) from SLN CRX-601 formulation study-1. CAT SLN: cationic SLNs, SLN-1.5%: SLNs prepared at 1.5% w/v of the carrier lipid, SLN-5%: SLNs prepared at 5% w/v of the carrier lipid. The values 1 μg or 5 μg refer to CRX-601 dosage/animal/vaccination.

FIG. 3: Post-tertiary serum HAI titers from sublingual evaluation of SLN/chitosan CRX-601 formulations study-1. All formulations tested were able to generate functional antibodies. The SLN-5%/CRX-601 and SLN/CRX-601+chitosan groups were able to generate antibodies at levels similar to control IM group. Statistical analysis: One-way ANOVA analysis of variance and Tukey post test.

FIG. 4: Post-secondary serum IgG titers (top), post-tertiary serum IgG titers (middle) and post-tertiary tracheal/vaginal wash IgA titers (bottom) from SLN CRX-601 formulation study-2. CAT SLN: cationic SLNs, SLN-1.5%: SLNs prepared at 1.5% w/v of the carrier lipid, SLN-5%: SLNs prepared at 5% w/v of the carrier lipid. CRX-601 dose is 5 μg/animal/vaccination in all SL treatment groups and 1 μg/animal/vaccination in the IM control.

FIG. 5: Post-tertiary serum HAI titers from sublingual evaluation of SLN/chitosan CRX-601 formulations study-2. All formulations tested were able to generate functional antibodies at levels generally similar to control IM groups (except Blk Cat SLN (SA-0.5%) old and BLK CAT SLN (SA-1%). Statistical analysis: One-way ANOVA analysis of variance and Tukey post test.

### DETAILED DESCRIPTION OF THE INVENTION

### Definitions

The term “solid lipid nanoparticle” (SLN) when used herein refers to a carrier in the submicron size range comprising lipids and surfactants and having a solidified lipid core. The lipids are typically biocompatible and biodegradable and solid at room and body temperature.

When used herein, the term “AGP” refers to aminoalkyl glucosaminide phosphates. This application discloses a number of examples of AGPs and includes references to further documents describing AGPs. Even further AGPs and information on the synthesis of AGPs can e.g. be found in Johnson et al. (1999) Bioorg Med Chem Lett 9: 2273.

AGPs used in the present invention are preferably TLR4 agonists, i.e. compounds which activate a TLR4 receptor to produce a biological response.

The term “mucoadhesive”, when used herein, refers to a tendency for adhesion to mucus or mucus membranes. “Transmucosal administration” refers to administration of a substance through mucus or mucus membranes.

### Further Aspects and Embodiments of the Invention

As explained above, in one aspect, the invention relates to a composition comprising solid lipid nanoparticles (SLNs), wherein the SLNs comprise an aminoalkyl glucosaminide phosphate (AGP).

### AGPs for Use in the Invention

In preferred embodiments, the AGP used in the invention is a TLR4 agonist.

AGPs suitable for use in the invention are disclosed in, for example, WO9850399, WO0134617, WO0212258, WO3065806, WO04062599, WO06016997, WO0612425, WO03066065, WO0190129 and WO2012055981, US 2003/0199460 and US 2005/0227943, the disclosure of which is herein incorporated by reference. Such molecules have also been described in the scientific and patent literature as lipid A mimetics.

In one aspect, the AGP is one in which an aminoalkyl (aglycon) group is glycosidically linked to a 2-deoxy-2-amino-a-D-glucopyranose (glucosaminide). The compounds are phosphorylated at the 4 or 6 carbon on the glucosaminide ring. Further, the compounds possess three 3-alkanoyloxyalkanoyl residues comprising a primary and secondary fatty acyl chain, each carbon chain consisting of from 2-24 carbon atoms, and preferably from 7-16 carbon atoms. In a preferred embodiment, each primary chain contains 14 carbon atoms and each secondary chain has between 10 and 14 carbon atoms.

In one embodiment, the AGP compounds are described by the general formula:

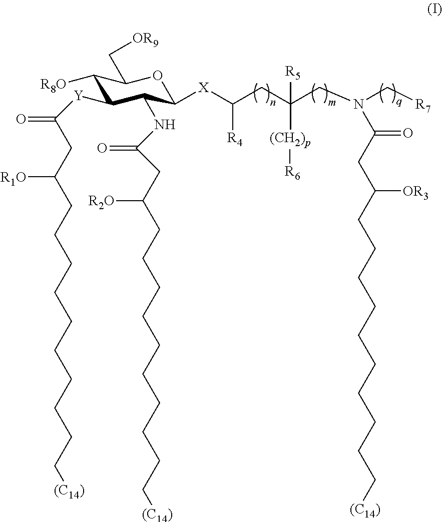

Such compounds comprise a 2-deoxy-2-amino-a-D-glucopyranose (glucosamine) in glycosidic linkage with an aminoalkyl (aglycon) group. Compounds are phosphorylated at the 4 or 6 carbon on the glucosamine ring and have three alkanoyloxyalkanoyl residues. The compounds are described generally by Formula I, wherein X represents an oxygen or sulfur atom, Y represents an oxygen atom or NH group, “n”, “m”, “p” and “q” are integers from 0 to 6, R1, R2, and R3 represent normal fatty acyl residues having 7 to 16 carbon atoms, R4 and R5 are hydrogen or methyl, R6 and R7 are hydrogen, hydroxy, alkoxy, phosphono, phosphonooxy, sulfo, sulfooxy, amino, mercapto, cyano, nitro, formyl or carboxy and esters and amides thereof; R8 and R9 are phosphono or hydrogen. The configuration of the 3′ stereogenic centers to which the normal fatty acyl residues are attached is R or S, but preferably R. The stereochemistry of the carbon atoms to which R4 or R5 are attached can be R or S.

In a preferred embodiment, X is oxygen. The number of carbon atoms between heteroatom X and the aglycon nitrogen atom is determined by variables “n” and “m”. Variables “n” and “m” can be integers from 0 to 6. In a preferred embodiment, the total number of carbon atoms between heteroatom X and the aglycon nitrogen atom is from about 2 to about 6 and most preferably from about 2 to about 4. In a further preferred embodiment, R8 is phosphono and R9 is hydrogen.

The compounds are hexaacylated, that is, they contain a total of six fatty acid residues. The aminoalkyl glucosamine moiety is acylated at the 2-amino and 3-hydroxyl groups of the glucosamine unit and at the amino group of the aglycon unit with 3-hydroxyalkanoyl residues. In Formula I, these three positions are acylated with 3-hydroxytetradecanoyl moieties. The 3-hydroxytetradecanoyl residues are, in turn, substituted with normal fatty acids (R1-R3), providing three 3-n-alkanoyloxytetradecanoyl residues or six fatty acid groups in total.

The chain length of normal fatty acids R1-R3 can be from about 7 to about 16 carbons. Preferably, R1-R3 are from about 9 to about 14 carbons. The chain lengths of these normal fatty acids can be the same or different. Although only normal fatty acids are described, it is expected that unsaturated fatty acids (i.e. fatty acid moieties having double or triple bonds) substituted at R1-R3 on the compounds would produce biologically active molecules.

Specific examples of AGPs suitable for use in the invention include CRX-527 which is disclosed in Stöver et al (2004) J Biol Chem 279, 6, page 4440. WO0212258 and WO3065806 disclose additional suitable embodiments of AGPs having a cyclic aminoalkyl (aglycon) linked to a 2-deoxy-2-amino-a-D-glucopyranose (glucosaminide), commonly referred to as “cyclic AGPs.” Reference generally to AGPs herein includes both cyclic and non cyclic AGPs.

Cyclic AGPs possess three 3-alkanoyloxyalkanoyl residues comprising a primary and secondary fatty acyl chain, each carbon chain consisting of from 2-24 carbon atoms, and preferably from 7-16 carbon atoms. In one preferred aspect each primary chain contains 14 carbon atoms and each secondary carbon chain has between 10 and 14 carbon atoms per chain.

The cyclic AGPs are described by the general formula II:

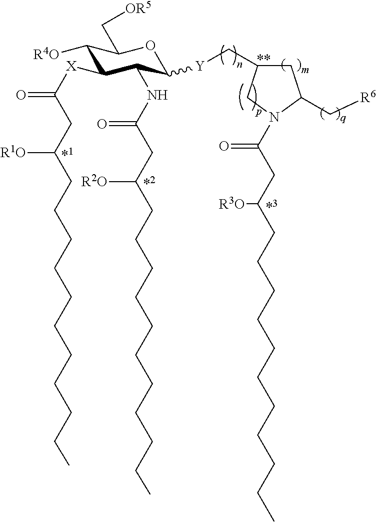

These compounds comprise a 2-deoxy-2-amino-p-D-glucopyranose (glucosamine) glycosidically linked to a cyclic aminoalkyl (aglycon) group. The compounds are phosphorylated at the 4 or 6-position of the glucosamine ring and acylated with alkanoyloxytetradecanoyl residues on the aglycon nitrogen and the 2 and 3-positions of the glucosamine ring. The compounds are described generally by formula (II): and pharmaceutically acceptable salts thereof, wherein X is —O— or NH— and Y is —O— or —S—; R1, R2, and R3 are each independently a (C2-C24) acyl group, including saturated, unsaturated and branched acyl groups; R4 is —H or —PO₃R7R8, wherein R7 and R8 are each independently H or (C1-C4) alkyl; R5 is —H, —CH₃ or —PO₃R9R10, wherein R9 and RI0 are each independently selected from —H and (CI-C4) alkyl; R6 is independently selected from H, OH, (CI-C4) alkoxy, —PO₃R11R12, —OPO₃R11R12, —SO₃R11, —OSO₃R11, —NR11R12, —SR11, —CN, —NO2, —CHO, —CO₂R11, and —CONR11R12, wherein R11 and R12 are each independently selected from H and (CI-C4) alkyl, wherein “*1-3” and “**” represent chiral centers; wherein the subscripts n, m, p and q are each independently an integer from 0 to 6, with the proviso that the sum of p and m is from 0 to 6.

In some embodiments, the cyclic AGP compound contains an —O— at X and Y, R4 is PO₃R7R8, R5 and R6 are H, and the subscripts n, m, p, and q are integers from 0 to 3. In a more preferred embodiment, R7 and R8 are —H. In an even more preferred embodiment, subscript n is 1, subscript m is 2, and subscripts p and q are 0. In yet an even more preferred embodiment, R1, R2, and R3 are tetradecanoyl residues. In a still more preferred embodiment, *1-3 are in the R configuration, Y is in the equatorial position, and ** is in the S configuration (N—[(R)-3-tetradecanoyloxytetradecanoyl]-(S)-2-pyrrolidinomethyl 2-deoxy-4-0-phosphono-2-[(R)-3-tetradecanoyloxytetradecanoylamino]-3-0-[(R)-3-tetradecanoyloxytetradecanoyl]-p-D-glucopyranoside and pharmaceutically acceptable salts thereof).

Preferred cyclic structures include:

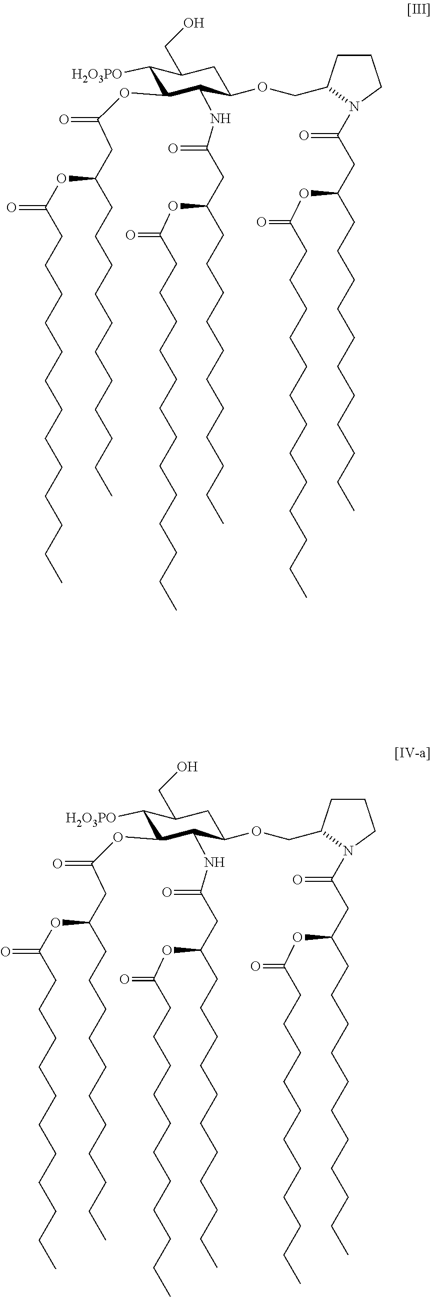

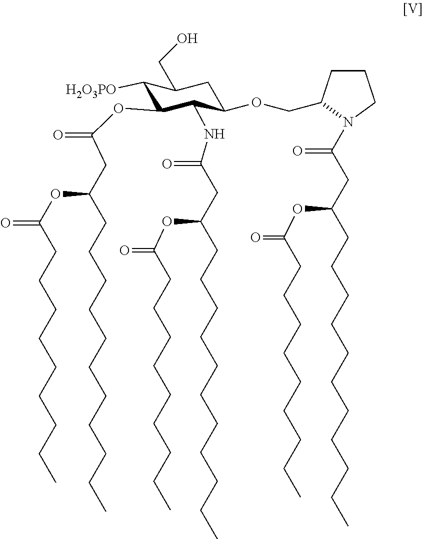

### Formula V is CRX 590.

In other embodiments, the AGP has one or more ether linked rather than ester linked primary and/or secondary lipid groups. In such embodiments, R1-R3 represent straight chain alkyl groups and not acyl groups, making the groups R1O—, R2O—, and R3O— alkoxy rather than alkanoyloxy groups and the attachment to the primary acyl chain an ether rather than an ester linkage. In the case of an ether-linked primary lipid group, the 3-alkanoyloxyalkanoyl residue attached to the 3-hydroxy group of the glucosamine unit is replaced with either a 3-alkanoyloxyalkyl moiety or a 3-alkoxyalkyl moiety, making the attachment of the primary lipid group to the glucosamine 3-position an ether rather than an ester linkage. A general formula for ethers is that of formula IV of WO2006016997.

An example of a preferred compound is CRX-601 (formula VI). In preferred embodiments, the composition comprises between, 0.1 and 1.5% (w/v) of CRX-601, more preferably between 0.1 and 1%, even more preferably between 0.2 and 0.5%, such as between 0.2 and 0.25%, or between 0.5 and 1%.

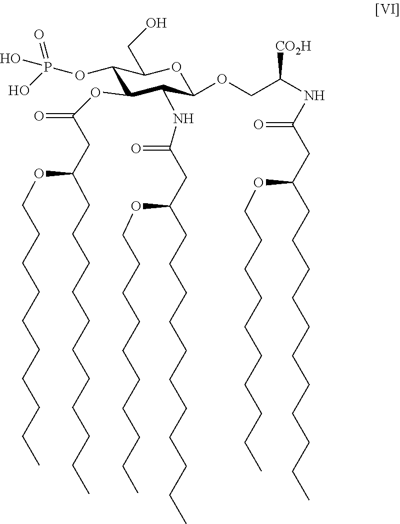

In other embodiments, the AGP molecule may have a different number of carbons in the molecule's primary chains and/or secondary chains. Such compounds are disclosed in WO04062599 and WO06016997. As with other AGPs, each carbon chain may consist of from 2-24 carbon atoms, and preferably from 7-16 carbon atoms. In one preferred embodiment, each primary chain contains 14 carbon atoms and each secondary carbon chain has between 10 and 14 carbon atoms per chain.

Such compounds are represented by the following structures VII, VIII, IX and X:

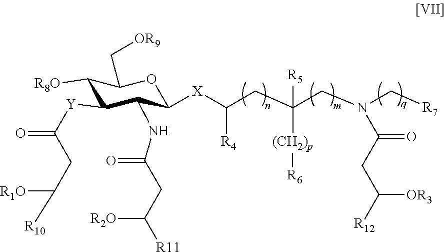

wherein X is selected from the group consisting of 0 and S at the axial or equatorial position; Y is selected from the group consisting of 0 and NH; n, m, p and q are integers from 0 to 6; R1, R2 and R3 are the same or different and are fatty acyl residues having from 1 to about 20 carbon atoms and where one of R1, R2 or R3 is optionally hydrogen; R4 and R5 are the same or different and are selected from the group consisting of H and methyl; R6 and R7 are the same or different and are selected from the group consisting of H, hydroxy, alkoxy, phosphono, phosphonooxy, sulfo, sulfooxy, amino, mercapto, cyano, nitro, formyl and carboxy, and esters and amides thereof; R8 and R9 are the same or different and are selected from the group consisting of phosphono and H, and at least one of R8 and R9 is phosphono; R10, R11 and R12 are independently selected from straight chain unsubstituted saturated aliphatic groups having from 1 to 10 carbon atoms; or a pharmaceutically acceptable salt thereof.

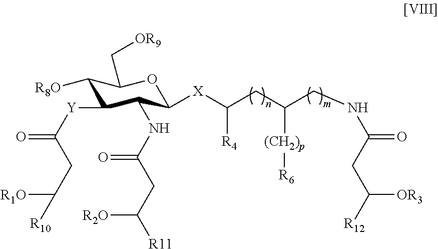

wherein X is selected from the group consisting of 0 and S at the axial or equatorial position; Y is selected from the group consisting of 0 and NH; n and m are 0; R1, R2 and R3 are the same or different and are fatty acyl residues having from 1 to about 20 carbon atoms and where one of R1, R2 or R3 is optionally hydrogen; R4 is selected from the group consisting of H and methyl; p is 1 and R6 is COOH or p is 2 and R6 is OP0₃H₂; R8 and R9 are the same or different and are selected from the group consisting of phosphono and H, and at least one of R8 and R9 is phosphono; and R10, R11 and R12 are independently selected from straight chain unsubstituted saturated aliphatic groups having from 1 to 10 carbon atoms; or a pharmaceutically acceptable salt thereof.

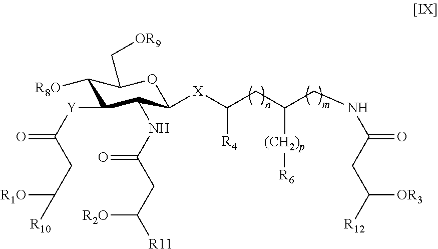

wherein X is selected from the group consisting of 0 and S at the axial or equatorial position; Y is selected from the group consisting of 0 and NH; n, m, p and q are integers from 0 to 6; R1, R2 and R3 are the same or different and are straight chain saturated aliphatic groups (i.e., straight chain alkyl groups) having from 1 to about 20 carbon atoms and where one of R1, R2 or R3 is optionally hydrogen; R4 and R5 are the same or different and are selected from the group consisting of H and methyl; R6 and R7 are the same or different and are selected from the group consisting of H, hydroxy, alkoxy, phosphono, phosphonooxy, sulfo, sulfooxy, amino, mercapto, cyano, nitro, formyl and carboxy, and esters and amides thereof; R8 and R9 are the same or different and are selected from the group consisting of phosphono and H, and at least one of R8 and R9 is phosphono; R10, R11 and R12 are independently selected from straight chain unsubstituted saturated aliphatic groups having from 1 to 11 carbon atoms; or a pharmaceutically acceptable salt thereof.

The general formula may also comprise an R5 group, at the same position as shown in formula VII above, wherein R5 is selected from the group consisting of H and methyl.

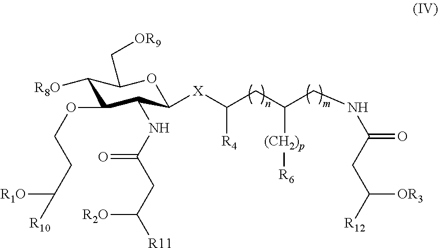

Yet another type of compound of this invention has the formula above (IV): wherein Y is now fixed as oxygen; X is selected from the group consisting of 0 and S at the axial or equatorial position; n and m are 0; R1, R2 and R3 are the same or different and are fatty acyl residues having from 1 to about 20 carbon atoms and where one of R1, R2 or R3 is optionally hydrogen; R4 is selected from the group consisting of H and methyl; p is 0 or 1 and R6 is COOH, or p is 1 or 2 and R6 is OPO3H2; R8 and R9 are the same or different and are selected from the group consisting of phosphono and H, and at least one of R8 and R9 is phosphono; and R10, R11 and R12 are independently selected from straight chain unsubstituted saturated aliphatic groups having from 1 to 10 carbon atoms; or a pharmaceutically acceptable salt thereof. These compounds thus have two acylated chains and one non-acylated ether chain.

Other suitable AGP structures such as CRX-524 are disclosed in Cluff et al. (2005) Infection and Immunity 73:3044.

Processes for making AGPs are also disclosed e.g. in WO0612425.

### Lipids and Surfactants Suitable for Use in the Invention

As explained above, SLNs are carriers in the submicron size range comprising lipids and surfactants and having a solidified lipid core. The lipids used are typically biocompatible and biodegradable and solid at room and body temperature.

Many lipids can be used for SLN preparation, and examples of suitable lipids are e.g. given Table 8.1 of Svilenov and Tzachev (2014) Nanomedicine Chapter 8, page 187 (ISBN (eBook): 978-1-910086-01-8: Publisher: One Central Press (OCP)) (herein incorporated by reference).

Preferred lipids for use in the present invention are behenates of glycerol, i.e. monobehenate of glycerol, dibehenate of glycerol and tribehenate of glycerol. Preferred are also mixtures of these three. Preferred concentrations are from 1 to 5% (w/v), such as from 1 to 4%, e.g. 1.5% or 3%. A preferred lipid is Compritol 888 ATO (glyceryl dibehenate European Pharmacopoeia [EP], glyceryl behenate National Formulary [NF]), which is a hydrophobic mixture of mono- (12-18% w/w), di- (45-54% w/w) and tri- (28-32% w/w) behenate of glycerol with melting point in range of 69-74° C. and with hydrophilic lipophilic balance (HLB)≈2.

Furthermore, many surfactants can be used for SLN preparation, and examples of suitable surfactant are e.g. given Table 8.2 of Svilenov and Tzachev (2014) Nanomedicine Chapter 8, page 187 (ISBN (eBook): 978-1-910086-01-8: Publisher: One Central Press (OCP)) (herein incorporated by reference). A preferred surfactant is polysorbate 80 (also known as Tween 80). Preferably, the composition comprises from 0.5 to 3% (v/v) polysorbate 80.

### Mucoadhesion

In preferred embodiments, the compositions of the invention are mucoadhesive. SLNs can be rendered more mucoadhesive by combination with or incorporation of mucoadhesive compounds.

In some embodiments, SLNs of the invention are cationic. Cationic SLNs can e.g. potentially allow mucoadhesion by their electrostatic interaction with polyanionic mucin coating on the sublingual mucosa. Preferably, the Zeta potential, e.g. as measured by dynamic light scattering is 20 mV or more, e.g. 20 to 30 mV, or more. SLNs can be rendered more cationic by combination with or incorporation of cationic lipids and/or cationic surfactants. Thus, in some embodiments, the SLNs in the composition of the invention comprise a cationic lipid and/or a cationic surfactant. In one embodiment, the SLNs comprise a compound selected from the group consisting of stearylamine (Octadecylamine) (SA), dimethyldioctadecylammonium bromide (DDAB), or cetyltrimethylammonium bromide (CTAB). In a further embodiment, the composition comprises 0.25-1.5% SA, DDAB or CTAB, such as 0.25-0.55%, e.g. 0.5% SA, DDAB or CTAB.

In some embodiments, the SLNs are rendered mucoadhesive by addition of a mucoadhesive agent. Preferred mucoadhesive agents are chitosan derivates, such as alkylated chitosans. A particularly preferred mucoadhesive agent is as methylglycol chitosan, which is preferably used in a concentration of 0.2 to 20 mg/ml, more preferably from 2 to 10, such as from 2 to 5 mg/ml.

### Further Preferred Properties of the Compositions of the Invention

In preferred embodiments, the composition of the invention has a size distribution below the sterile filterability limit, to avoid loss of material during sterile filtration. Thus, in a preferred embodiment, the average size of the SLNs in the composition is between 30 and 200 nm, such as between 30 and 150 nm, such as between 30 and 100 nm. Furthermore, in a preferred embodiment, the polydispersity index is less than 0.75, such as less than 0.5, such as less than 0.4. Polydispersity index and size may e.g. be measured using a Malvem Zetasizer.

In a further preferred embodiment, the composition remains a stable, homogeneous, composition and does not become a semisolid gel for at least 24 hrs following its preparation.

### Methods for Preparing Compositions of the Invention

Methods for preparing SLNs are described in the art. Such methods have e.g. been described in Svilenov and Tzachev (2014) Nanomedicine Chapter 8, page 187 (ISBN (eBook): 978-1-910086-01-8: Publisher: One Central Press (OCP)) (herein incorporated by reference).

As explained above, in an even further aspect, the invention relates to a method for preparing a composition comprising SLNs as described herein, said method comprising:
a. Dissolving and mixing AGP and a lipid in an organic solvent
b. Removing the organic solvent from the mixture
c. Heating the resulting mixture and mixing with an aqueous solution containing a surfactant to generate an oil-in-water mixture, and
d. Performing a size reduction step, for example using microfluidization or sonication, and
e. Allowing the composition to cool
   The method may include a filtration step.

### Uses of Compositions of the Invention

Compositions of the invention may be used in medicine. The compositions may be used on their own or in combinations with other substances, for instance in combination with an antigen for use in immunization.

Methods for preventing and treating diseases, e.g. infectious diseases, autoimmune diseases or allergies, by administering AGPs in the absence of exogenous antigens are disclosed in WO03066065 and WO0190129. Compositions of the invention may be used in such methods.

As mentioned above, compositions of the invention may also be used as adjuvants in vaccination. Thus, in a further aspect, the invention relates to an immunogenic composition comprising an SLN composition as described herein and an antigen. Vaccine preparation is generally described in Vaccine Design (“The subunit and adjuvant approach” (eds Powell M. F. & Newman M.J.) (1995) Plenum Press New York). The compositions of the invention are suitable for combination with many different antigens, including e.g. proteins, nucleic acids and polysaccharides of various origins. In one embodiment, the antigen is an influenza antigen, such as haemagglutinin.

The compositions or immunogenic compositions of the invention may be used to protect or treat a mammal by means of administering via systemic or transmucosal route. These administrations may include injection via the intramuscular, intraperitoneal, intradermal or subcutaneous routes; or via transmucosal administration to the oral/alimentary, respiratory, genitourinary tracts. Administration via a transmucosal route, such as sublingual administration is preferred. The composition of the invention may be administered as a single dose, or multiple doses.

Subject doses of the composition described herein typically range from about 0.1 μg to 10 mg of AGP per administration. Preferred mucosal or local doses range from 1 μg to 10 mg of AGP per administration, and most typically 10 μg to 1 mg of AGP.

The disclosure will be further described by reference to the following, non-limiting, examples:

### EXAMPLES

### Example 1: Preparation of SLNs with CRX-601

Stock solutions of CRX-601 (2% w/v) and Compritol 888 ATO (glyceryl behenate: a mixture of mono-, di-, and tri-behenate of glycerol; 5% w/v) were prepared in tetrahydrofuran (THF). Variable amounts of these stock solutions were added to a glass vial so as to obtain the desired concentrations of CRX-601 and Compritol 888 ATO in the final aqueous preparation (shown in Table 1). The organic solvent was removed by evaporation on a rotary evaporator and further with high pressure vacuum for 12 hrs. The CRX-601 lipid mix thus obtained was heated to >80° C., and to it an aqueous solution containing Tween 80 (0.25-2% v/v) was added at the same temperature under high speed mixing (8000-12000 rpm). The resultant oil-in-water emulsion was sonicated further (15-25 min) using a probe sonicator on a hot plate maintained at a temperature >80° C. The SLN composition was then allowed to congeal at room temperature and aseptically filtered using a 0.22 μm filter into a sterile depyrogenated container. Representative average particle sizes and zeta-potential values of the resultant formulations as measured by dynamic light scattering are shown in Table 1.

The aminoalkyl glucosaminide 4-phosphate (AGP) CRX-601 used in this work was synthesized as described previously (Bazin et al. (2008) Bioorg & Med Chem Lett 18: 5350 (CRX-601 is the ether analog of CRX-527)) and purified by chromatography (to >95% purity). CRX-601, either in the starting material or in the final product was quantified by a standard reverse phase HPLC analytical method.

Formulations with higher than 5% w/v Compritol and >1% w/v CRX-601 yield nanoparticles with a size distribution approaching the sterile filterability limit, and are associated with loss of material during sterile filtration.

TABLE 1
Representative formulation parameters for CRX-601 SLNs.

| | | Average | | |
|---|---|---|---|---|
| | Sonication | particle | | Zeta- |
| | time | size | | potential |
| Formulation Composition | (min) | (nm) | PDI | (mV) |
| 1.5% glyceryl behenate/2% | 15 | ~35 | 0.24 | ~−31 |
| tween 80/0.45 mg/mL CRX-601 | | | | |
| 1.5% glyceryl behenate/2% | 15 | ~35 | 0.40 | ~−42 |
| tween 80/0.9 mg/mL CRX-601 | | | | |
| 1.5% glyceryl behenate/2% | 15 | ~40 | 0.45 | ~−43 |
| tween 80/1.2 mg/mL CRX-601 | | | | |
| 1.5% glyceryl behenate/2% | 15 | ~50 | 0.27 | ~−48 |
| tween 80/1.5 mg/mL CRX-601 | | | | |
| 3% glyceryl behenate/2% | 15 | ~90 | 0.39 | ~−40 |
| tween 80/1.5 mg/mL CRX-601 | | | | |
| 6% glyceryl behenate/2% | 15 | ~130 | 0.22 | NT |
| tween 80/1.5 mg/mL CRX-601 | | | | |
| 10% glyceryl behenate/2% | 15 | ~400 | 0.29 | NT |
| tween 80/1.5 mg/mL CRX-601 | | | | |
| 1.5% glyceryl behenate/2% | 15 | ~40 | 0.39 | ~−46 |
| tween 80/2.5 mg/mL CRX601 | | | | |
| 6% glyceryl behenate/2% | 15 | ~120 | 0.18 | ~−49 |
| tween 80/2.5 mg/mL CRX601 | | | | |
| 1.5% glyceryl behenate/2% | 25 | ~180 | 0.57 | ~−40 |
| tween 80/2 mg/mL CRX-601 | | | | |
| 1.5% glyceryl behenate/0.5% | 25 | ~117 | 0.24 | −15 |
| tween 80/2 mg/mL CRX-601 | | | | |
| 1.5% glyceryl behenate/0.75% | 25 | ~82 | 0.20 | −32 |
| tween 80/2 mg/mL CRX-601 | | | | |
| 1.5% glyceryl behenate/1% | 15 | ~82 | 0.48 | ~−18 |
| tween 80/2 mg/mL CRX-601 | | | | |
| 1.5% glyceryl behenate/2% | 25 | ~56 | 0.27 | ~−34 |
| tween 80/2 mg/mL CRX-601 | | | | |
| 3% glyceryl behenate/2% | 15 | ~90 | 0.26 | ~−28 |
| tween 80/2 mg/mL CRX-601 | | | | |
| 3% glyceryl behenate/2% | 25 | ~95 | 0.43 | ~−14 |
| tween 80/2 mg/mL CRX-601 | | | | |
| 1.5% glyceryl behenate/3% | 15 | ~200 | 0.55 | ~−18 |
| tween 80/2 mg/mL CRX-601 | | | | |

### Comparative Example

Stearic acid was explored for preparation of SLNs (instead of glyceryl behenate). Stearic acid concentrations of 1.5%, 3.0% and 6.0% were used. CRX-601 was targeted at 1, 2 and 3 mg/mL. Clear homogeneous formulations were initially seen right after preparation, but upon storage overnight turned into semisolid gels.

### Example 2: Preparation of Cationic CRX-601/SLNs

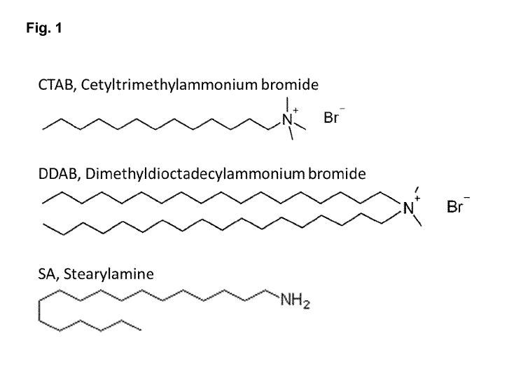

SLN formulations based on glyceryl behenate are anionic in zeta-potential. Cationic SLNs were prepared by incorporation of cationic lipids/surfactants (shown in FIG. 1) into the glyceryl behenate SLNs. The cationic SLNs could potentially allow mucoadhesion by their electrostatic interaction with polyanionic mucin coating on the sublingual mucosa. The formulations were prepared as in Example 1, but with addition of 0.1-1% w/v stearylamine (Octadecylamine) (SA), dimethyldioctadecylammonium bromide (DDAB), or cetyltrimethylammonium bromide (CTAB). Representative average particle sizes and zeta-potential values of the resultant formulations as measured by dynamic light scattering are shown in Table 2.

TABLE 2
Summary of formulation parameters for cationic SLNs.

| | 1.5% glyceryl | | 6% glyceryl | |
|---|---|---|---|---|
| | behenate-SLN | | behenate-SLN | |
| | Avg size | ZP | Avg size | ZP |
| Cationic lipid/surfactant | (nm)/PDI | (mV) | (nm)/PDI | (mV) |
| Control, no cationic lipid | 51/0.18 | −15 | ~130/0.2 | ~−30 |
| 0.1% SA | 84/0.26 | +30 | 153/0.23 | +51 |
| 0.5% SA | 79/0.18 | +38 | 189/0.25 | +52 |
| 1.0% SA | 72/0.28 | NT | NT | NT |
| 0.1% DDAB | 55/0.25 | +26 | 137/0.22 | +50 |
| 0.5% DDAB | 51/0.24 | +25 | 130/0.22 | +49 |
| 0.1% CTAB | 53/0.37 | +22 | 89/0.40 | +56 |
| 0.5% CTAB | 40/0.37 | +34 | 77/0.30 | +50 |
| 0.05% SA, 1 mg/mL | 54/0.32 | −15 | NT | NT |
| CRX-601 | | | | |
| 0.05% SA, 2 mg/mL | 82/0.89 | −39 | NT | NT |
| CRX-601 | | | | |
| 0.1% SA, 2 mg/mL CRX-601 | 46/0.27 | −26 | 80/0.25 | −23 |
| 0.5% SA, 1 mg/mL CRX-601 | 108/0.19 | +44 | NT | NT |
| 0.5% SA, 2 mg/mL CRX-601 | 383/0.23 | +37 | NT | NT |
| 0.05% DDAB, 1 mg/mL | 49/0.24 | −27 | NT | NT |
| CRX-601 | | | | |
| 0.05% DDAB, 2 mg/mL | 119/0.93 | −50 | NT | NT |
| CRX-601 | | | | |
| 0.1% DDAB, 2 mg/mL | 54/0.25 | −26 | 107/0.20 | −32 |
| CRX-601 | | | | |
| 0.5% DDAB, 1 mg/mL | 37/0.47 | +40 | NT | NT |
| CRX-601 | | | | |
| 0.5% DDAB, 2 mg/mL | 53/0.44 | +54 | NT | NT |
| CRX-601 | | | | |

NT: Not tested

Only the highest tested cationic lipid concentration of 0.5% gave cationic SLNs, and not the lower concentrations, probably due to the excess of anionic charge contributed by CRX-601 compared to in the blank SLN preparations. Higher DDAB wt. % of 0.6, 0.8 and 1% were further tested but in all cases a PDI of >0.4 was obtained with multimodal size distribution. Hence, SA based cationic SLNs with 1.5% glyceryl behenate have been selected for initial testing in mouse sublingual study.

### Example 3: Preparation of CRX-601/SLNs Coated with Methylglycol Chitosan

Methylglycol chitosan (chitosan glycol trimethyl ammonium iodide) was dissolved in 10 mM HEPES or 10 mM HEPES-saline buffer pH 7.2 to yield concentrations of 2 and 10 mg/ml. The solutions were aseptically filtered using a 0.22 μm filter into a sterile depyrogenated container. The formulations from Examples 1 and 2 were mixed aseptically with varying volumes of methylglycol chitosan solution to yield concentrations of methylglycol chitosan ranging from 1-10 mg/mL. Representative average particle sizes and zeta-potential as measured by dynamic light scattering are shown in Table 3. In some formulation combinations, where the PDI values were exceeding 0.3 (bimodal or multimodal size distribution), sonication of the formulation on a water bath sonicator for 10 min was used to reduce PDI values and make the size distribution unimodal. Overall, the data indicates some increase in particle size and a reversal in zeta-potential (net positive potential from a net negative potential) exceeding approximately 1 mg/mL methylglycol chitosan, consistent with surface coating with methylglycol chitosan. At concentrations exceeding a certain threshold, methylglycol chitosan is expected to be saturating the SLN surface, with excess being free in solution.

TABLE 3
Summary of formulation parameters for methylglycol
chitosan coated SLN-CRX-601.

| MGC | | | | | | |
|---|---|---|---|---|---|---|
| Concentration | 10 mM HEPES | | | 10 mM HEPES-Saline | | |
| (CRX-601 conc | Avg size | ZP | Distri- | Avg size | ZP | Distri- |
| ~1 mg/mL) | (nm)/PDI | (mV) | bution | (nm)/PDI | (mV) | bution |

CRX-601/1.5%-SLN

| | | | | | | |
|---|---|---|---|---|---|---|
| Control/no | 45/0.25 | −33 | U | 38/0.20 | −9 | U |
| chitosan | | | | | | |
| 0.2 mg/mL | NT | NT | | 38/0.13 | −6 | U |
| 0.4 mg/mL | 50/0.14 | NT | U | 42/0.15 | −15 | U |
| 0.6 mg/mL | 50/0.17 | −14 | U | 52/0.14 | −1.3 | U |
| 0.8 mg/mL | 95/0.19 | +4 | U | 64/0.17 | −1.8 | U |
| 1.0 mg/mL | 75/0.20 | +14 | U | 73/0.22 | +3.5 | U |
| 2.5 mg/mL | 87/0.35 | +48 | U | 64/0.27 | +16 | U |
| 5.0 mg/mL | 92/0.33 | +33 | U | 67/0.35 | +20 | U |

CRX-601/6%-SLN

| | | | | | | |
|---|---|---|---|---|---|---|
| Control/no | 114/0.22 | −42 | U | 110/0.16 | −27 | U |
| chitosan | | | | | | |
| 0.2 mg/mL | 112/0.16 | −39 | U | 118/0.21 | −25 | U |
| 0.4 mg/mL | 116/0.16 | −23 | U | 167/0.30 | −20 | U |
| 0.6 mg/mL | 138/0.24 | −22 | U | 240/0.32 | −15 | U |
| 0.8 mg/mL | 385/0.50 | −9 | M | 530/0.53 | −6 | U |
| 1.0 mg/mL | 720/0.46 | −4 | M | 313/0.30 | −2 | B |
| 2.5 mg/mL | 405/0.45 | +50 | B | 326/0.30 | +9 | B |
| 5.0 mg/mL | 373/0.27 | +35 | U | 110/0.16 | +48 | B |

NT: Not tested,
U: Unimodal,
B: Bimodal,
M: Multimodal

Most formulation combinations were unimodal in size distribution. 1.5%-SLN formulations were below 100 nm, while 6%-SLN formulations were >200 nm. No precipitation/aggregation was observed at any of the tested MGC concentrations indicating incorporation of CRX-601 into SLNs (precipitation is typically observed with free aqueous CRX-601 in the zone of neutrality).

### Comparative Example

An alternate formulation preparation method based on the study by Sandri et al., [J of Microencapsulation, 2010, 27 (8), 735-746], which describes preparation of SLNs directly in chitosan solution (instead of water used earlier) was also attempted. Only blank SLNs (without CRX-601) were prepared, representative average particle sizes and zeta-potential measurements for which are shown in Table 4.

TABLE 4
Summary of formulation parameters for methylglycol chitosan
associated SLNs prepared using method of Sandri et al.

| | 10 mM HEPES | | | 10 mM HEPES-Saline | | |
|---|---|---|---|---|---|---|
| MGC | Avg size | ZP | | Avg size | ZP | |
| Concentration | (nm)/PDI | (mV) | Distribution | (nm)/PDI | (mV) | Distribution |

1.5% glyceryl behenate-SLN

| | | | | | | | |
|---|---|---|---|---|---|---|---|
| 2.5 | mg/mL | 540/0.41 | 36 | B/M | 5310/1.0 | 21 | U/turbid |
| 5 | mg/mL | 221/0.44 | 55 | B | 2000/1.0 | 32 | B |
| 10 | mg/mL | 680/0.6 | NT | M | 797/0.59 | 34 | B |

6% glyceryl behenate-SLN

| | | | | | | | |
|---|---|---|---|---|---|---|---|
| 2.5 | mg/mL | 7490/0.5 | 0.34 | M/turbid | 193/0.59 | 20 | B |
| 5 | mg/mL | 570/0.27 | 55 | U | NT | NT | NT |
| 10 | mg/mL | 560/0.24 | 63 | U | 300/0.41 | 46 | B |

U: Unimodal,
B: Bimodal,
M: Multimodal,
NT: not tested

Most formulations had considerably higher PDI values with this method (Table 4), hence this method was not considered a suitable method for preparation of CRX-601/SLNs.

### Example 4: Rabbit Pyrogen Test

The pyrogen test is used here as a surrogate measure of CRX-601 incorporation into SLNs from Example 1-3 and as a measure of their stability in biological milieu. The test was performed at Pacific Biolabs (Hercules, Calif.) as per their SOP 16E-02, which follows procedures outlined in USP<151>. The 1.5% SLN/CRX-601 formulation lacked pyrogenicity at a concentration of 25 ng CRX-601/kg animal body weight. This lack of pyrogenicity up to 25 ng/kg corresponding to an at least 10 fold improvement over free CRX-601 (max non-pyrogenic dose of 1-2 ng/kg), and indicates a >90% incorporation of CRX-601 into the SLNs. The individual temperature increases from three rabbits per test are indicated in table 5.

TABLE 5
Representative rabbit pyrogen test measurements for
some of the formulations described in Example 1, 2,
and 3. Values in parenthesis are maximum temperature
change for three animals during the testing period.

| | Assay outcome NP* or P* (Individual temperature | | |
|---|---|---|---|
| | change in ° C. for three rabbits) | | |
| | CRX-601 dose: | CRX-601 dose: | CRX-601 dose: |
| Formulation | 2.5 ng/kg | 25 ng/kg | 50 ng/kg |
| CRX-601/ | NP (0.0, | NP (0.1, | P (0.7, |
| 1.5%-SLN | 0.0, 0.0) | 0.1, 0.3) | 0.5, 0.6) |
| CRX-601/ | NP (0.0, | NP (0.1, | P (0.8, |
| 5%-SLN | 0.0, 0.0) | 0.4, 0.3) | 0.4, 0.9) |
| CRX-601/ | NP (0.0, | NP (0.0, | P (0.6, |
| 3%-SLN | 0.1, 0.0) | 0.2, 0.3) | 0.6, 0.7) |
| CRX-601/ | NT | NT | P (1.2, |
| cationic SLN | | | 0.8, 0.5) |

NT: not tested; *NP: Non-pyrogenic response, P: Pyrogenic response. The test is considered NP if none of the rabbits shows an individual temperature rise of 0.5° C. or more above its respective control temperature at any point.

### Example 5: Compatibility of Flu Antigen with SLN Formulations

Compatibility with split Flu antigen (A/Victoria/210/2009 H3N2, lot#AS20APA-051-02) was tested by admixing with the SLN formulations at concentrations required for animal dosing. The resultant combination was tested for any precipitation by visual observation and by turbidometry (any decrease in % transmitted light at 600 nm). No significant precipitation or change in % T was observed with any of the formulations.

### Example 6: Mouse Sublingual Vaccination and Determination of Specific Antibody Responses

Female BALB/c mice (6 to 8 weeks of age) obtained from Charles River Laboratories (Wilmington, Mass.) were used for these studies. Mice anesthetized by intraperitoneal (i.p) administration of ketamine (100 mg/kg) and xylazine (10 mg/kg) were given vaccine by sublingual administration (5-6 μLs). All mice were vaccinated on days 0, 21 and 42 with the 5 μg CRX-601 in the SLN formulation admixed with 1 or 1.5 μg HA/mouse using the influenza antigen A/Victoria/210/2009 H3N2. Serum was harvested using retro orbital puncture on day 36 (14dp2) under anesthesia, on day 56 (14dp3) mice were sacrificed and a final harvest of vaginal washes, tracheal washes and serum were collected. All animals were used in accordance with guidelines established by the U.S. Department of Health and Human Services Office of Laboratory Animal Welfare and the Institutional Animal Care and Use Committee at GSK Biologicals, Hamilton, Mont.

Specific antibody responses were measured by two independent immunoassays, the enzyme linked immunosorbent assay (ELISA) and the influenza hemagglutinin inhibition (HI) assay.

ELISA was performed using split flu coated 96 well plates (Nunc Maxisorp) and detecting the bound immunoglobins from the added serum or tracheal wash or vaginal wash samples using peroxidase linked goat anti-mouse IgG, IgG1, IgG2a or IgA. This was followed by addition of an enzyme specific chromogen, which resulted in color intensity directly proportional to the amount of specific antiflu IgGs/IgAs contained in the serum. The optical density was read at 450 nm.

HI assay was performed by evaluating inhibition of chicken or rooster RBCs upon exposure to flu virus in presence of mouse serum. The reciprocal of the last dilution of influenza virus which resulted in complete or partial agglutination of RBCs was used to calculate the HI titer and expressed as HA units/50 μl of sera.

### Example 7: Mouse Sublingual Vaccination with SLN Formulations (NIH #157, 165, 170, 171)

Based on above described studies with various SLN formulations, the formulations described in table 6 below were selected, prepared, characterized, sterile filtered, and quantitated by RP-HPLC for a mouse sublingual study (study-1).

TABLE 6
Summary of formulations prepared for mouse sublingual study (study-1).

| | | Z-Ave | | | Conc. By |
|---|---|---|---|---|---|
| | | size | | ZP | RP-HPLC |
| Formulation | SLN composition | (nm) | PDI | (mV) | (mg/mL) |
| Blank | | | | | |
| Blank cationic SLN | 1.0% glyceryl behenate, 0.5% stearyl amine, | 69 | 0.28 | +52 | NA |
| (Blk CAT SLN) | 2% tween 80 | | | | |
| Blank SLN (5% SLN) | 5% glyceryl behenate, 2% tween 80 | 140 | 0.17 | −18 | NA |
| Blank chitosan | 1.5% glyceryl behenate, 2% tween 80, | 67 | 0.31 | +27 | NA |
| coated SLN | 5 mg/mL methylglycol chitosan | | | | |
| (1.5% SLN/Chito) | | | | | |
| Cationic SLN/CRX-601 | 1.0% glyceryl behenate, 0.5% stearyl amine, | 77 | 0.26 | +35 | 0.97 |
| (CAT SLN CRX601) | 2% tween 80, 0.97 mg CRX-601/mL | | | | |
| SLN-1.5%/CRX-601 | 1.5% glyceryl behenate, 2% tween 80, | 69 | 0.23 | −42 | 2.3 |
| | 2.3 mg CRX-601/mL | | | | |
| SLN-5%/CRX-601 | 5% glyceryl behenate, 2% tween 80, | 116 | 0.15 | −40 | 1.2 |
| | 1.2 mg CRX-601/mL | | | | |
| Chitosan coated | 1.5% glyceryl behenate, 2% tween 80, | 125 | 0.26 | +41 | 1.15 |
| SLN/CRX-601 | 5 mg/mL methylglycol chitosan, 1.15 mg | | | | |
| | CRX-601/mL | | | | |
| CRX601-DOPC | | | | | |
| CRX601 Aqueous | | | | | |
| intramuscular | | | | | |

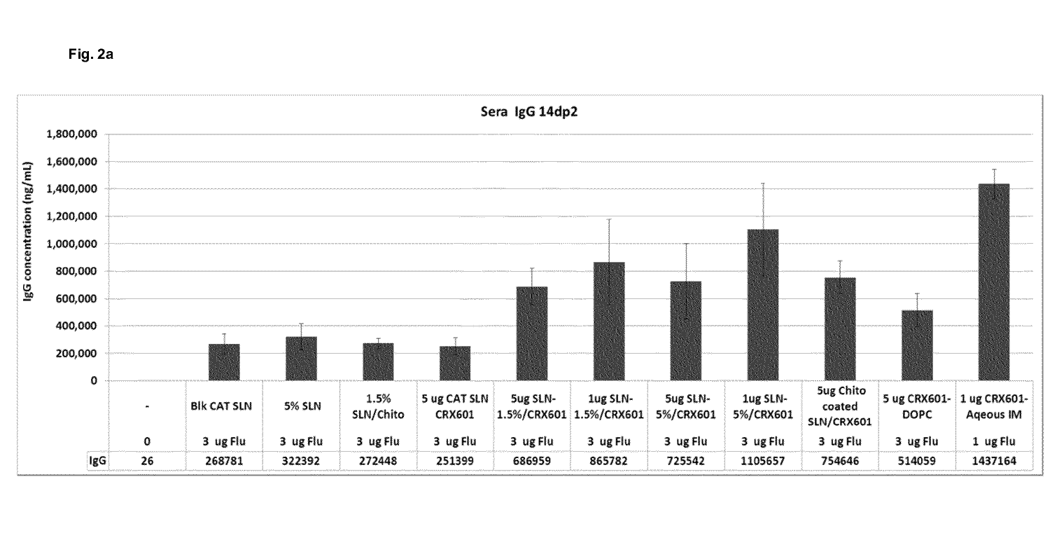

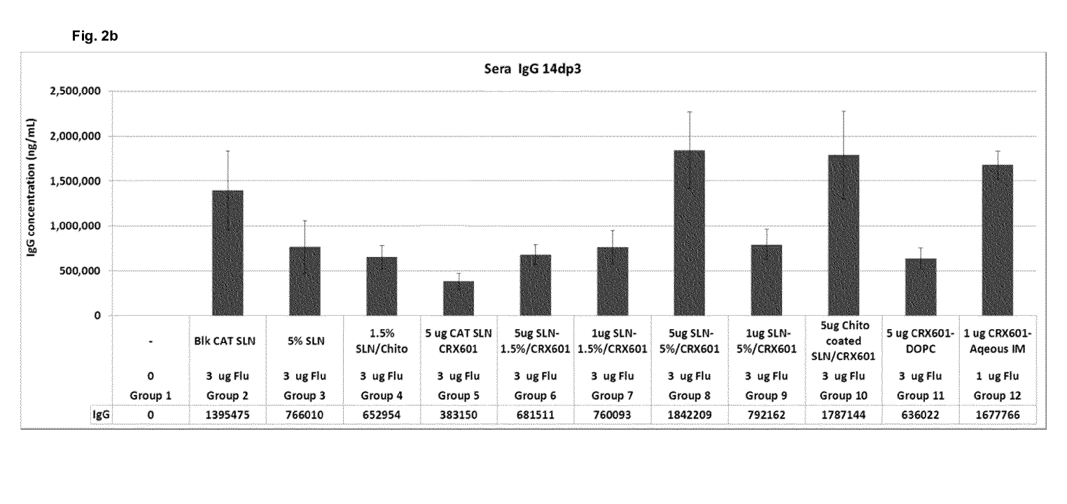

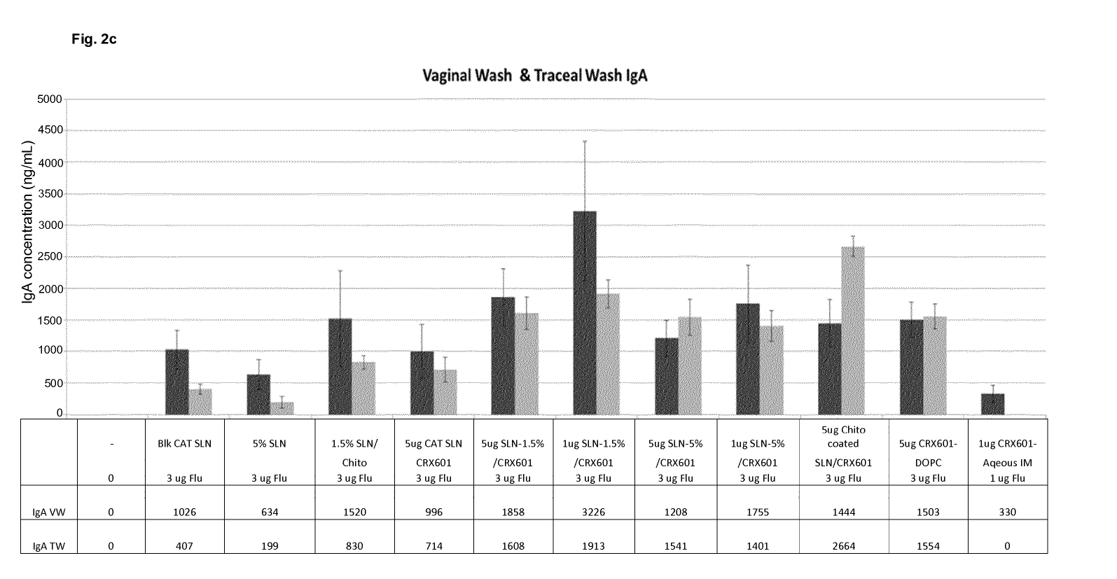

The mice were vaccinated using the procedure outlined in Example 6 with formulations from table 6. Post-secondary serum antibody titers were higher with SLN formulations of CRX-601 compared to vehicle controls or a CRX-601 DOPC (1,2-dioleoyl-sn-glycero-3-phosphocholine) liposome formulation (FIG. 2, top panel), with the exception of cationic SLN CRX-601, which was not better than the controls in this particular assay. Mice vaccinated via intramuscular administration of CRX-601+split flu antigen are also included as a positive control. For most groups of mice, post-tertiary serum antibody titers were boosted when compared to post-secondary titers (FIG. 2, middle panel). The pattern of titers was somewhat different from post-secondary, with 5 μg CRX-601 in 5%-SLN and 5 μg CRX-601 in chitosan coated 1.5%-SLN providing the best overall titers. Surprisingly, blank cationic SLNs also provided a strong humoral immunity which could be due to adsorption of the anionic flu antigen on the surface of these nanoparticles, aiding in their transport across the SL mucosa. Anti-flu IgA titers were readily detected in mucosal washes from mice vaccinated SL with split flu and SLN formulations or CRX-601 controls (FIG. 2).

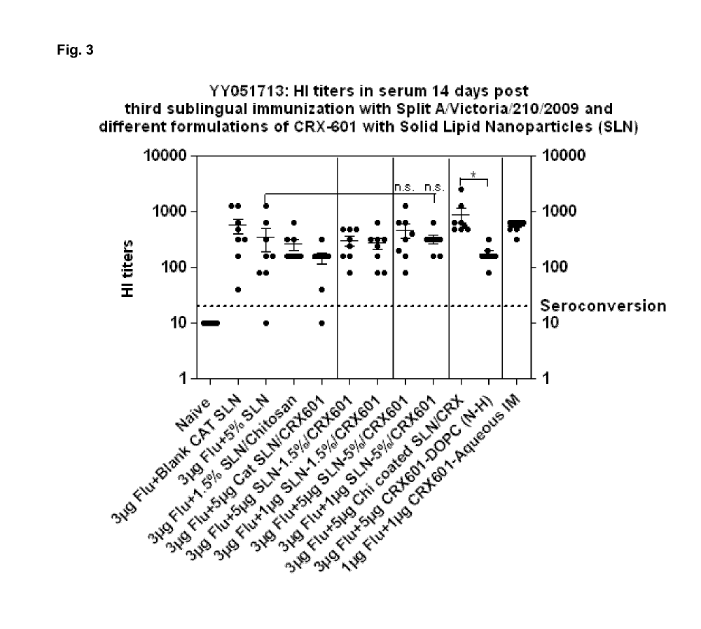

Serum HI titers were highest in mice vaccinated with chitosan coated 1.5%-SLN 5 μg CRX-601 group and significantly higher than the CRX-601 DOPC liposome group (FIG. 3). No significant differences were noted between other SL treatment groups.

The study was followed up a SL study-2, with formulations described in table 7, with mice vaccinated using the procedure outlined in Example 6. The follow up study includes additional groups with blank cationic SLNs, additional ratio of SLN-1.5%/CRX-601+MGC, and additional group with SLN-5%/CRX-601+MGC formulation compositions.

TABLE 7
SLN formulations prepared for mouse sublingual study-2. All formulations
were prepared at a CRX-601 concentration >1 mg/mL and were further
diluted to 1 mg/mL before transfer to immunology.

| | | Z-Ave size | ZP |
|---|---|---|---|
| Formulation | SLN composition | (nm)/PDI | (mV) |
| Blank | | | |
| BLK CAT SLN | Blank cationic SLNs containing 0.5% Stearylamine, | 69/0.28 | +52 |
| (SA-0.5%) | retest of the previous batch | | |
| BLK CAT SLN | Blank cationic SLNs containing 0.5% Stearylamine | 94/0.20 | +11 |
| (SA-0.5%) | | | |
| BLK CAT SLN | Blank cationic SLNs containing 1.0% Stearylamine | 72/0.28 | +0.5 |
| (SA-1%) | | | |
| BLK CAT SLN | Blank cationic SLNs (SA-0.5%) with methylglycol | NT | NT |
| (SA-0.5%)/Chit 5 | chitosan (5 mg/mL) | | |
| SLN-1.5%/CRX-601 | SLNs prepared with 1.5% glyceryl behenate/CRX-601 | 65/0.6* | −30 |
| SLN-1.5%/CRX-601/ | Methylglycol chitosan (5 mg/mL) coated SLNs prepared | 56/0.27 | +13 |
| Chit 5 | with 1.5% glyceryl behenate/CRX-601 | | |
| SLN-1.5%/CRX-601/ | Methylglycol chitosan (10 mg/mL) coated SLNs prepared | 52/0.28 | +7 |
| Chit 10 | with 1.5% glyceryl behenate/CRX-601 | | |
| SLN-5%/CRX-601 | SLNs prepared with 5% glyceryl behenate/CRX-601 | 120/0.22 | −30 |
| SLN-5%/CRX-601/ | Methylglycol chitosan (5 mg/mL) coated SLNs prepared | NT | NT |
| Chit 5 | with 5% glyceryl behenate/CRX-601 | | |
| Flu only | | | |
| intramuscular | | | |
| CRX601 Aqueous | | | |
| intramusclar | | | |

*Although the PDI value is much higher than previously observed, the formulation was clear, did not indicate any aggregation and was therefore deemed acceptable for further use.
NT: Not tested.

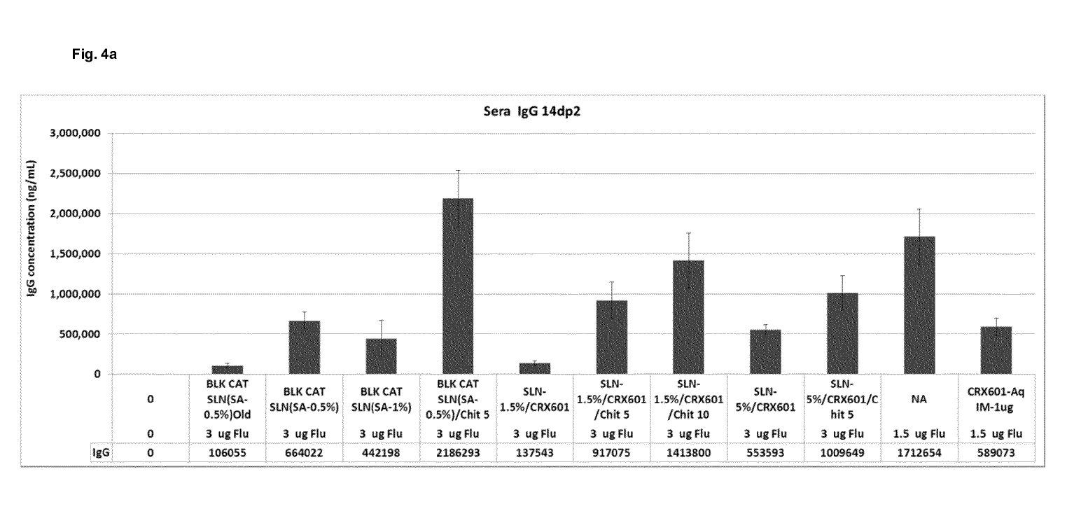

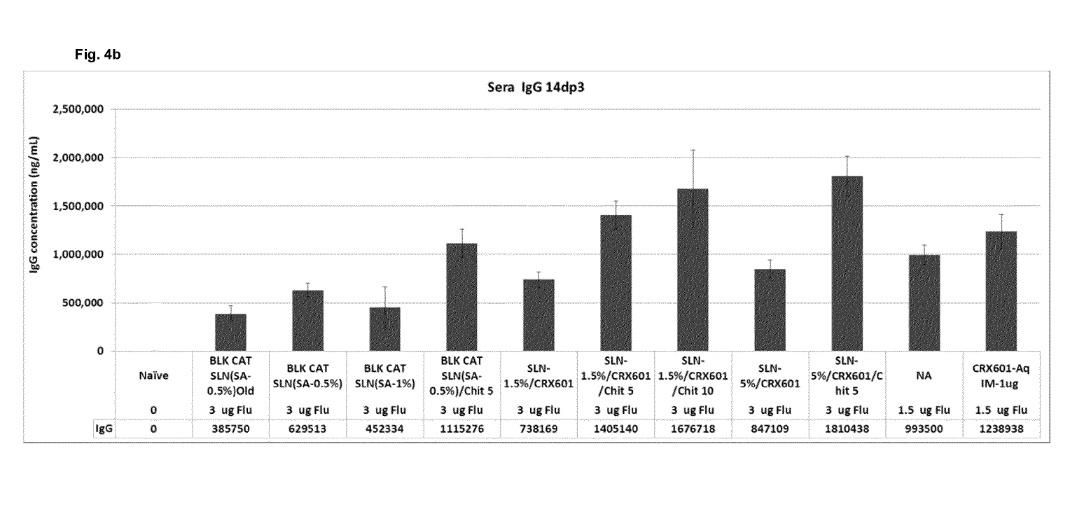

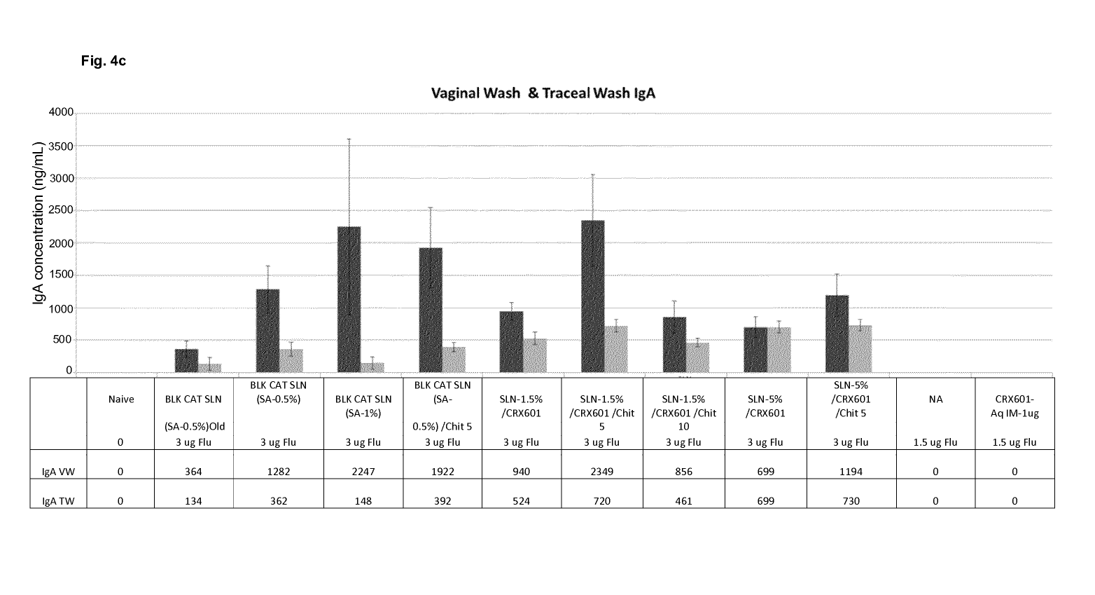

The results from study-2 are summarized in FIG. 4. In general, serum IgG responses were greater when chitosan was present with the SLN formulation, relative to the matched SLN formulation without chitosan (FIG. 4). CRX-601 1.5% and 5% SLN formulations with chitosan had IgG responses greater than the IM control groups. Notably, CAT SLN formulations without CRX-601 elicited significant levels of IgG (FIG. 4). Post-secondary serum antibody trends were similar to post-tertiary, although with lower levels. All sublingual vaccine groups demonstrated significantly higher tracheal and vaginal wash IgA titres versus the IM positive controls, with vaginal wash titers outperforming tracheal wash titers (FIG. 4). CRX-601 1.5% SLN with 5 mg/mL chitosan elicited the greatest IgA response, in comparison to other CRX-601 formulations. Notably, CAT SLN formulations without CRX-601 (BLK) also elicited significant levels of IgA.

Based on the results from this study, SLN formulations, particularly with 5-10 mg/mL chitosan, are promising lead candidates for SL vaccination formulations. In addition, CAT SLN formulations without CRX-601 elicited significant levels of IgG and IgA.

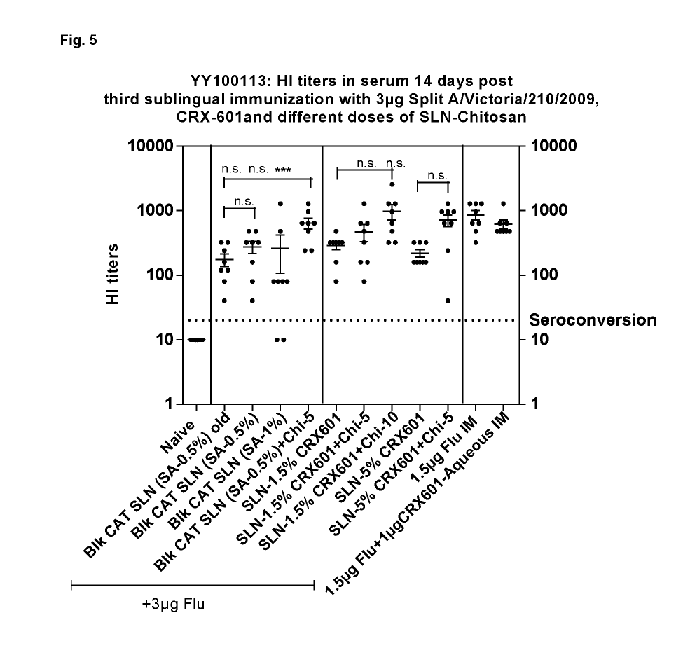

Post-tertiary HI titers corresponded to post-tertiary IgG titers. All formulations tested were able to generate functional antibodies at levels generally similar to control IM groups (except Blk CAT SLN (SA-0.5%) old and BLK CAT SLN (SA-1%) (FIG. 5).

1. A composition comprising solid lipid nanoparticles (SLNs), wherein the SLNs comprise an aminoalkyl glucosaminide phosphate (AGP).

2. The composition of claim 1, wherein the AGP is a TLR4 agonist.

3. The composition of claim 2, wherein the AGP is CRX-601:

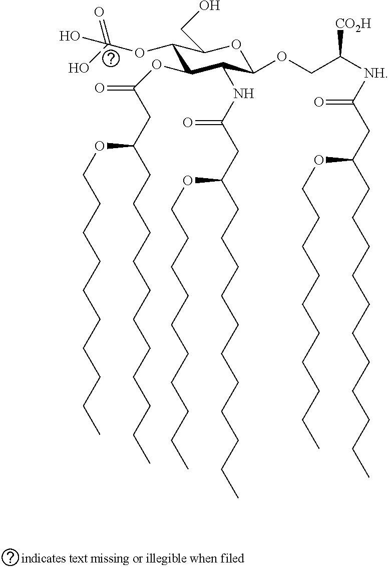

4. The composition of claim 3, wherein the composition comprises from 0.45 to 2.5 mg/ml CRX-601.

5. The composition of claim 1, wherein the SLNs comprise glyceryl behenate.

6. The composition of claim 5, wherein the composition comprises from 1 to 5% (w/v) glyceryl behenate, such as 3% (w/v) glyceryl behenate.

7. The composition of claim 1, wherein the SLNs comprise polysorbate 80.

8. The composition of claim 7, wherein the composition comprises from 0.5 to 3% (v/v) polysorbate 80.

9. The composition of claim 1, wherein the SLNs are cationic.

10. The composition of claim 9, wherein the SLNs comprise a cationic lipid and/or a cationic surfactant.

11. The composition of claim 1, wherein the SLNs comprise a compound selected from the group consisting of SA, DDAB or CTAB.

12. The composition of claim 11, wherein the composition comprises 0.25-1.5% SA, such as 0.25-0.55% SA, e.g. 0.5% SA or 0.25-1.5% DDAB, such as 0.25-0.55% DDAB, e.g. 0.5% DDAB.

13. The composition of claim 1, wherein the Zeta potential is 20 mV or more, e.g. between 20 and 30 mV.

14. The composition of claim 1, wherein the composition further comprises a mucoadhesive agent, such as methylglycol chitosan.

15. (canceled)

16. The composition of claim 1, wherein the average size of the SLNs in the composition is between 30 and 200 nm, such as between 30 and 150 nm, such as between 30 and 100 nm.

17. The composition of any one of the preceding claims, wherein the polydispersity index of the SLNs is less than 0.75, such as less than 0.5, such as less than 0.4.

18. An immunogenic composition comprising an SLN composition according to claim 1 and an antigen.

19. The immunogenic composition of claim 18, wherein the antigen is an influenza antigen, such as haemagglutinin.

20. (canceled)

21. (canceled)

22. The composition of claim 1, wherein the composition is administered via a transmucosal route, such as sublingual administration.

23. A method for preparing a composition comprising SLNs according to claim 1, said method comprising:
a. Dissolving and mixing AGP and a lipid in an organic solvent
b. Removing the organic solvent from the mixture
c. Heating the resulting mixture and mixing with an aqueous solution containing a surfactant to generate an oil-in-water mixture
d. Performing a size reduction step, for example using microfluidization or sonication, and
e. Allowing the composition to cool.

24. (canceled)

25. (canceled)
